(19) United States
(12) Patent Application Publication
(10) Pub. No.: US 2023/0061212 A1
(43) Pub. Date: Mar. 2, 2023

(54) CRYOGENIC PROBE INLET COUPLING

(71) Applicant: AtriCure, Inc., Mason, OH (US)

(72) Inventors: Drew Douglas Schierloh, Glandorf, OH (US); Justin David Volz, West Chester, OH (US); Brian Frederick Metcalf, Springdale, OH (US); Stefan Stefanov, Ludlow, KY (US)

(73) Assignee: AtriCure, Inc., Mason, OH (US)

(21) Appl. No.: 17/461,201

(22) Filed: Aug. 30, 2021

(51) Int. Cl. F16L 19/075

(52) U.S. Cl. CPC F16L 19/075

(57) ABSTRACT

The present disclosure pertains to cryogenic devices and related methods. A ferrule coupling according to at least some aspects of the present disclosure may include a male fitting including a bore; a female fitting including a bore, the female fitting sized to receive the male fitting; a conduit sized to fit within the bores of the male fitting and the female fitting, wherein a circumferential gap exists between an exterior of the conduit and an interior surface of the female fitting demarcating the bore; a first ferrule including dual conical surfaces and a cylindrical bore, the cylindrical bore sized to receive the conduit; a second ferrule including dual conical surfaces and a cylindrical bore, the cylindrical bore sized to receive the conduit; and/or a washer including dual conical surfaces and a cylindrical bore, the cylindrical bore sized to receive the conduit.

### INTRODUCTION TO THE INVENTION

The present disclosure is directed to pressurized sealed fittings and couplings that may be used as part of medical and surgical instruments and devices and related methods, and, more specifically, to components of cryogenic devices and related methods.

The present disclosure contemplates that cryogenic devices, such as cryogenic probes, may be used in various medical and surgical procedures. Generally, cryogenic probes may be used to apply extremely cold temperatures to a target tissue. Cryogenic probes may be used for cryoablation and/or cryoanalgesia, for example.

The present disclosure contemplates that some cryogenic probes may be supplied with one or more cryogenic fluids, which may be used to cool a tissue-contacting portion, such as an ablation tip. Some cryogenic probes may include supply conduits, which convey cryogenic fluid to the ablation tip, and exhaust conduits, which convey used cryogenic fluid away from the ablation tip. Some cryogenic probes may utilize one or more joints to connect the supply conduits and/or exhaust conduits of the cryogenic probes to respective supply and exhaust conduits (e.g., tubing and/or hoses) extending between the cryogenic probes and sources of cryogenic fluid and/or receptacles for used cryogenic fluid. Some cryogenic probes may utilize cryogenic fluids supplied at high pressures. For example, some cryogenic probes employing Joule-Thompson expansion in the ablation tip may receive liquid nitrous oxide at up to about 1200 psi and about room temperature and/or may exhaust the nitrous oxide a gas or mixed phase of gas and liquid at about 45 psi and about −90° F. The joints between the cryogenic probe and the tubing or hoses may be designed to withstand such pressures and temperatures.

The following patent references may provide context for the present disclosure and are incorporated by reference herein in their entireties: U.S. Pat. No. 8,915,908, U.S. Patent Application Publication No. 2012/0109117, U.S. Patent Application Publication No. 2020/0022745, and U.S. Patent Application Publication No. 2020/0085485.

While known devices have been used safely and effectively to connect cryogenic probes to supply and/or exhaust tubing and hoses, improvements in the construction and operation of fittings for cryogenic devices may be beneficial for users (e.g., surgeons) and patients. The present disclosure includes various improvements which may enhance the construction, operation, and methods of use of cryogenic devices.

It is an aspect of the present disclosure to provide a ferrule coupling including a male fitting including a bore; a female fitting including a bore, the female fitting sized to receive the male fitting; a conduit sized to fit within the bores of the male fitting and the female fitting, wherein a circumferential gap exists between an exterior of the conduit and an interior surface of the female fitting demarcating the bore; a first ferrule including dual conical surfaces and a cylindrical bore, the cylindrical bore sized to receive the conduit; a second ferrule including dual conical surfaces and a cylindrical bore, the cylindrical bore sized to receive the conduit; and/or a washer including dual conical surfaces and a cylindrical bore, the cylindrical bore sized to receive the conduit.

In a detailed embodiment, the interior surface of the female fitting may delineate a cylindrical cavity section transitioning into a conical cavity section. An interior surface of the male fitting may delineate a conical cavity section. The conical section of the female fitting may be adjacent a section having an exposed helical cavity. The male fitting may include an exposed helical thread configured to be received within the exposed helical cavity and convert rotational motion between the male and female fittings into longitudinal motion between the male and female fittings.

In a detailed embodiment, the conical section of the female fitting may be sized to receive the first ferrule. A first of the dual conical surfaces of the first ferrule may be configured to engage the interior surface of the conical section of the female fitting. The conical section of the male fitting may be sized to receive the second ferrule. A first of the dual conical surfaces of the second ferrule may be configured to engage the interior surface of the conical section of the male fitting.

In a detailed embodiment, a second of the dual conical surfaces of the first ferrule may be configured to engage a first of the dual conical surfaces of the washer. A second of the dual conical surfaces of the second ferrule may be configured to engage a second of the dual conical surfaces of the washer.

In a detailed embodiment, the first ferrule may include a frustoconical exterior shape; the second ferrule may include a frustoconical exterior shape; the conical cavity section of the annual female fitting may have a frustoconical shape; and/or the conical cavity section of the annual male fitting may have a frustoconical shape. The conical cavity section of the annual male fitting may taper in an opposite direction than does the conical cavity section of the female fitting when the male fitting is received and coupled to the female fitting. The frustoconical exterior shape of the first ferrule may taper in an opposite direction than does the frustoconical exterior shape of the second ferrule when the male fitting is received and coupled to the female fitting.

In a detailed embodiment, the first ferrule may include a frustoconical interior cavity; the second ferrule may include a frustoconical interior cavity; the washer may include at least two exterior surfaces each having a frustoconical shape; and/or the dual conical surfaces of the washer may taper in opposite directions. The frustoconical interior cavity of the first ferrule may taper in the same direction as does the frustoconical shape of a first of the at least two exterior surfaces of the washer when the first ferrule and the washer are adjacent one another and both circumscribe the conduit. The frustoconical interior cavity of the second ferrule may taper in the same direction than does the frustoconical shape of a second of the at least two exterior surfaces of the washer when the second ferrule and the washer are adjacent one another and both circumscribe the conduit. The washer may be wedged between the first ferrule and the second ferrule when: the first ferrule is wedged between the conduit and the female fitting; and/or the second ferrule is wedged between the conduit and the male fitting.

It is an aspect of the present disclosure to provide a ferrule coupling including an annular male fitting including a bore; an annual female fitting including a bore, the annular female fitting sized to receive the annular male fitting; a conduit sized to fit within the bores of the annular male fitting and the annular female fitting; a first ferrule circumscribing at least a first portion of the conduit, the first ferrule including dual conical surfaces; a second ferrule circumscribing at least a second portion of the conduit, the second ferrule including dual conical surfaces; and/or a washer circumscribing at least a third portion of the conduit, the washer interposing the first ferrule and the second ferrule, the washer including dual conical surfaces. The first ferrule, the second ferrule, and/or the washer may longitudinally interpose the annular male fitting and the annular female fitting. The first ferrule may radially interpose the annular male fitting and the conduit. The second ferrule and/or the washer may radially interpose the annular female fitting and the conduit.

It is an aspect of the present disclosure to provide a ferrule coupling including a pair of ferrules, each of the pair of ferrules including a through bore and a first external inclined surface, a first interior inclined surface, and a bore interior surface delineating the through bore, where an incline of the first external inclined surface is less than sixty degrees with respect to a longitudinal axis centered with respect to the through bore, and where an incline between of the first interior inclined surface is no greater than ninety degrees with respect to the longitudinal axis; and/or a washer configured to interpose the pair of ferrules and including a through bore, the washer including a first exterior inclined surface and a second exterior inclined surface, where the first exterior inclined surface of the washer is configured to engage the first external inclined surface of a first of the pair of ferrules, and where the second exterior inclined surface of the washer is configured to engage the first external inclined surface of a second of the pair of ferrules.

It is an aspect of the present disclosure to provide a method of creating a fluid tight seal across conduit sections including circumscribing a first conduit with a first ferrule, a first washer, and a second ferrule, where the first ferrule includes dual conical surfaces tapering in the same direction, and where the second ferrule includes dual conical surfaces tapering in the same direction; compressing the first ferrule between the first conduit and a first fitting to form first seals, where a first of the first seals is between the first ferrule and the conduit, and where a second of the first seals is between the first ferrule and the first fitting; compressing the second ferrule between the first conduit and a second fitting to form second seals, the second fitting including at least a portion of a second conduit, where a first of the second seals is between the second ferrule and the conduit, and where a second of the second seals is between the second ferrule and the second fitting; and/or coupling the first fitting to the second fitting to maintain the first seals and the second seals.

In a detailed embodiment, the act of compressing the first ferrule between the first conduit and the first fitting to form first seals may include swaging the conduit at a first circumferential location; the act of compressing the second ferrule between the first conduit and the second fitting to form second seals may include swaging the conduit at a second circumferential location; and/or the first circumferential location may be longitudinally spaced along a length of the conduit from the second circumferential location. Compressing the first ferrule between the first conduit and the first fitting may include longitudinally repositioning a washer, including dual frustoconical surfaces, along an exterior of the first conduit; and/or longitudinally repositioning the washer may include longitudinally repositioning the second ferrule along the exterior of the first conduit. Longitudinal repositioning of the washer and the second ferrule may be the result of longitudinal repositioning of the first fitting with respect to the second fitting. The first fitting and/or the second fitting may be configured to threadably engage one another. Longitudinal repositioning of the first fitting with respect to the second fitting may be the result of rotation of the first fitting with respect to the second fitting when the first and second fittings are threadably engaged.

It is an aspect of the present disclosure to provide a cryogenic surgical system including a coupling as described above; a cryogenic probe, the cryogenic probe comprising the conduit; a cryogenic fluid source; and/or a second conduit extending between the cryogenic fluid source and the coupling.

It is an aspect of the present disclosure to provide a cryogenic surgical system including a coupling as described above; a pair of fittings housing the couplings; a cryogenic probe in sealed fluid communication with a conduit extending through the coupling; a cryogenic fluid source; and/or a second conduit extending between the cryogenic fluid source and the coupling.

### BRIEF DESCRIPTION OF THE DRAWINGS

Example embodiments are described in conjunction with the accompanying drawing figures in which:

FIG. 1 is a detailed cross-sectional view of an example compression fitting;

FIG. 2 is a detailed cross-sectional view of an example ferrule set of the compression fitting of FIG. 1;

FIG. 3 is a detailed cross-sectional view of an example coupling including the compression fitting of FIG. 1;

FIG. 4 is a simplified schematic view of an example cryogenic surgical system including the coupling of FIG. 3;

FIG. 5 is a detailed cross-sectional view of an alternative example coupling; and

FIG. 6 is detailed cross-sectional view of an example swaging tool; all in accordance with at least some aspects of the present disclosure.

### DETAILED DESCRIPTION

Example embodiments according to the present disclosure are described and illustrated below to encompass devices, methods, and techniques relating to sealing high pressure fluid carrying conduits that may be used as part of medical and surgical devices, as well as medical and surgical procedures. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure.

The present disclosure includes, among other things, high pressure sealed fittings and couplings for maintaining fluid-tight connections along fluid conveying components and, more specifically, to medical and surgical instruments and devices and related methods that incorporate high pressure sealed fittings and couplings as part of cryogenic devices and related methods. Some example embodiments according to at least some aspects of the present disclosure may be particularly useful in connection with connecting cryogenic probes to supply and/or exhaust tubing and/or hoses.

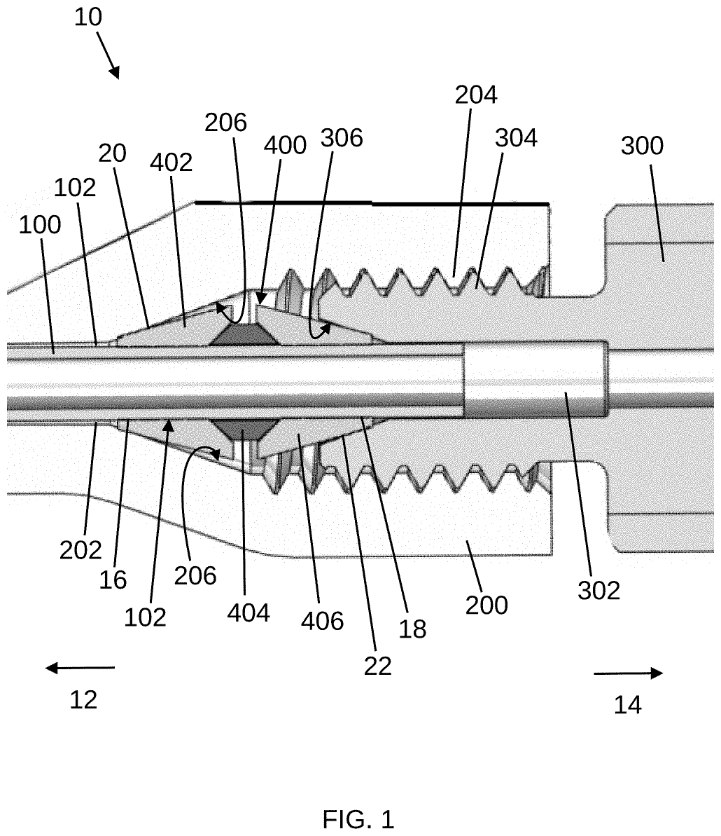

FIG. 1 is a detailed cross-sectional view of an example compression fitting 10, according to at least some aspects of the present disclosure. Generally, the compression fitting 10 may be configured to provide one or more pressure-tight (e.g., sealed) connections between a first fluid conduit 100, a first threaded component 200, and/or a second threaded component 300. The compression fitting 10 may include a ferrule set 400, which may be disposed on the outside of the first fluid conduit 100 and/or within at least one of the threaded components 200, 300.

For clarity, the following description references a first direction 12 and a second direction 14. The second direction 14 may be generally opposite the first direction 12. For example, in the context of a cryogenic fluid flowing from distal to proximal, the first direction 12 may be moving in a generally distal direction and/or the second direction 14 may be moving in a generally proximal direction. As used herein, “distal” may refer to a direction generally away from an operator (e.g., a surgeon) of a system or device, such as toward the distant-most end of a device that is inserted into a patient's body. As used herein, “proximal” may refer to a direction generally toward an operator of a system or device (e.g., a surgeon), such as away from the distant-most end of a device that is inserted into a patient's body. It is to be understood, however, that example directions referenced herein are merely for purposes of explanation and clarity, and should not be considered limiting.

In some example embodiments, the first fluid conduit 100 may include a tube, such as a stainless-steel tube. The first fluid conduit 100 may act as an inlet conduit of a cryogenic probe, for example. In some such embodiments, the first fluid conduit 100 may extend from generally distal to the fitting 10, through an inner cavity 202 of the first threaded component 200, and proximally at least partially into an interior cavity 302 of the second threaded component 300. In some example embodiments, the first threaded component 200 may be formed as a part of or attached to a manifold component. In further exemplary embodiments, the manifold component may be part of a cryogenic probe.

In some example embodiments, the first threaded component 200 may include an internally threaded portion 204 configured to threadedly engage an externally threaded portion 304 of the second threaded component 300. In some example embodiments, the internally threaded portion 204 and the externally threaded portion 304 may comprise UNF (Unified National Fine) thread, which may or may not form a fluid tight seal. In some example embodiments, the second threaded component 300 may be threaded into the first threaded component 200 generally in a relative distal direction. Generally, in various example embodiments, either of the threaded components 200, 300 may be externally threaded and the other internally threaded, and either threaded component 200, 300 may be positioned distally and the other proximally.

In various example embodiments, the threaded components 200, 300 may be constructed from any suitable materials, such as plastic(s) (e.g., PEEK (polyetheretherketone)) and/or metal(s) (e.g., stainless steel). In the example embodiment illustrated in FIG. 1, the first threaded component 200 may be constructed from PEEK and/or the second threaded component 300 may be constructed from stainless steel.

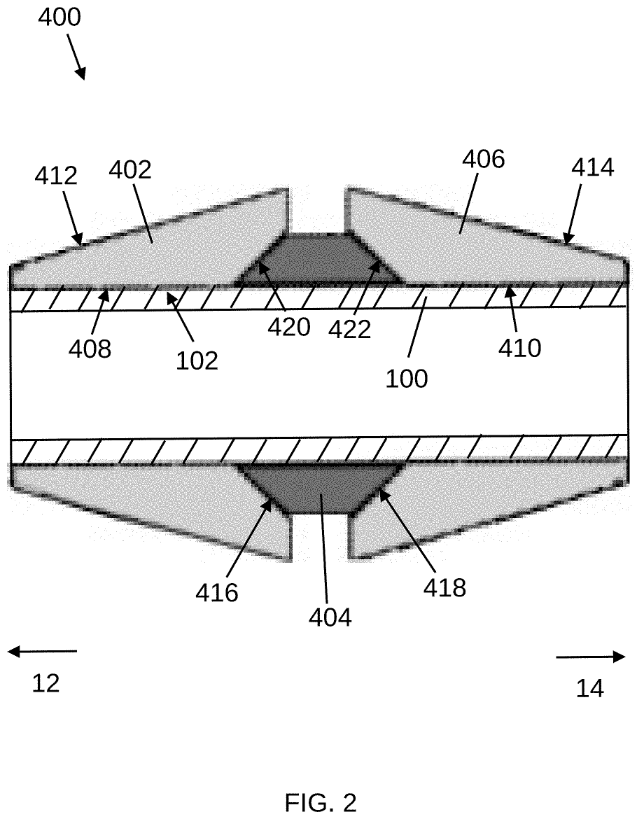

FIG. 2 is a detailed cross-sectional view of the example ferrule set 400, according to at least some aspects of the present disclosure. Referring to FIGS. 1 and 2, in some example embodiments, the ferrule set 400 may include a first tapered ferrule 402, a conically tapered washer 404, and/or a second tapered ferrule 406.

In some example embodiments, the ferrule set 400 may engage an outwardly facing surface 102 of the first fluid conduit 100. For example, the first ferrule 402 may be wedged or swaged onto the first fluid conduit 100 to form a sealed interface 16 between an inwardly facing surface 408 of the first ferrule 402 and the outwardly facing surface 102 of the first fluid conduit 100. Similarly, the second ferrule 406 may be wedged or swaged onto the first fluid conduit 100 to form a sealed interface 18 between an inwardly facing surface 410 of the second ferrule 406 and the outwardly facing surface 102 of the first fluid conduit 100.

In some example embodiments, the ferrule set 400 may engage inwardly facing surfaces of the first threaded component 200 and/or the second threaded component 300. For example, the first threaded component 200 may include an inwardly conically tapering, inwardly facing surface 206 (in the distal direction) and/or the first ferrule 402 may include an inwardly conically tapering, outwardly facing surface 412 (in the distal direction). The inwardly facing surface 206 of the first threaded component 200 may engage the outwardly facing surface 412 of the first ferrule 402 to form a sealed interface 20 therebetween. Similarly, the second threaded component 300 may include an inwardly conically tapering, inwardly facing surface 306 (in the proximal direction) and/or the second ferrule 406 may include an inwardly conically tapering, outwardly facing surface 414 (in the proximal direction). The inwardly facing surface 306 of the second threaded component 300 may engage the outwardly facing surface 414 of the second ferrule 406 to form a sealed interface 22 therebetween.

In some example embodiments, one or more of the sealed interfaces 16, 18, 20, 22 described above may provide one or more pressure-tight (e.g., sealed) connections between the first fluid conduit 100, a first threaded component 200, and/or a second threaded component 300. For example, the sealed interface 16 (between the first fluid conduit 100 and the first ferrule 402) and the sealed interface 20 (between the first ferrule 402 and the first threaded component 200) may provide a pressure-tight connection between the outwardly facing surface 102 of the first fluid conduit 100 and the inwardly conically tapering, inwardly facing surface 206 of the first threaded component 200. Accordingly, fluid present between the first fluid conduit 100 and the first threaded component 200 (e.g., outside of the first fluid conduit 100 and within the cavity 202) is prevented from traveling proximally beyond the sealed interfaces 16, 20. In some example cryogenic probes incorporating this example fitting 10, a cryogenic fluid exhaust path may be fluidically connected to the volume outside of the first fluid conduit 100 and within the cavity 202. Thus, the sealed interfaces 16, 20 may prevent leakage into and/or out of the cryogenic fluid exhaust path proximate the fitting 10.

Similarly, the sealed interface 18 (between the first fluid conduit 100 and the second ferrule 406) and the sealed interface 22 (between the second ferrule 406 and the second threaded component 300) may provide a pressure-tight connection between the outwardly facing surface 102 of the first fluid conduit 100 and the inwardly conically tapering, inwardly facing surface 306 of the second threaded component 300. Thus, fluid present between the first fluid conduit 100 and the second threaded component 300 (e.g., outside of the first fluid conduit 100 and within the cavity 302) is prevented from traveling distally beyond the sealed interfaces 18, 22. Accordingly, fluid present within the first fluid conduit 100 is prevented from leaking at the fitting 10. In some example devices incorporating this example fitting 10, such as a cryogenic probe, a fluid supply path may be fluidically connected to the interior cavity 302 of the second threaded component 300. Therefore, the sealed interfaces 18, 22 may prevent leakage into and/or out of the cryogenic fluid supply path proximate the fitting 10.

Referring to FIG. 2, in some example embodiments, the ferrule set 400 may be configured to facilitate the sealed interfaces 16, 18, 20, 22 (FIG. 1). For example, the tapered washer 404 may include opposed, outwardly facing, inwardly tapering, tapered surfaces 416, 418 configured to engage corresponding inwardly facing, inwardly conically tapering, tapered surfaces 420, 422 of the first ferrule 402 and the second ferrule 406, respectively.

In some example embodiments, the first tapered surface 420 of the tapered washer 404 may be disposed generally distally on the tapered washer 404. The first tapered surface 420 may taper inwardly in the distal direction. The second tapered surface 422 of the tapered washer 404 may be disposed generally proximally on the tapered washer 404. The second tapered surface 422 may taper inwardly in the proximal direction. Accordingly, the tapered washer 404 may include opposed, outwardly facing, inwardly tapering, tapered surfaces 420, 422.

In some example embodiments, the inwardly facing surface 416 of the first ferrule 402 may be disposed generally proximally on the first ferrule 402. The inwardly facing surface 416 may taper conically inwardly in the distal direction. The inwardly facing surface 418 of the second ferrule 406 may be disposed generally proximally on the second ferrule 406. The inwardly facing surface 418 may taper conically inwardly in the proximal direction. Accordingly, the first ferrule 402 may include a distal, outwardly facing, tapered surface 412 and a proximal, inwardly facing, tapered surface 420. Similarly, the second ferrule 406 may include a proximal, outwardly facing, tapered surface 414 and a distal, inwardly facing, tapered surface 422. In some example embodiments, the first ferrule 402 and the second ferrule 406 may be substantially identical, but may be assembled in the fitting 10 (FIG. 1) in opposite orientations. In some example embodiments, the ferrules 402, 406 may differ in at least one of size and/or shape, thus making them non-identical. In some example embodiments, the tapered washer 404 may be substantially symmetrical (e.g., the surfaces 416, 418 may be substantially mirror images of each other). In other example embodiments, the tapered washer 404 may be non-symmetrical.

Referring to FIGS. 1 and 2, in some example embodiments, the ferrule set 400 may be compressed longitudinally (e.g., proximal-distal) as the threaded components 200, 300 are threaded together (e.g., one or both of the threaded components 200, 300 are rotated with respect to one another). As the first ferrule 402 and the second ferrule 406 are pressed longitudinally towards one another, the tapered surfaces 420, 422 of the ferrules 402, 406 and the tapered surfaces 416, 418 of the tapered washer 404 may interact, such as to increase the engagement of the ferrules 402, 406 with the first fluid conduit 100 and/or the threaded components 200, 300. For example, portions of the ferrules 402, 406 proximate the tapered washer 404 may be driven radially outward (e.g., against the first threaded component 200 and/or the against the second threaded component 300). Portions of the ferrules 402, 406 away from the tapered washer 404 may be driven radially inward (e.g., against the first fluid conduit 100).

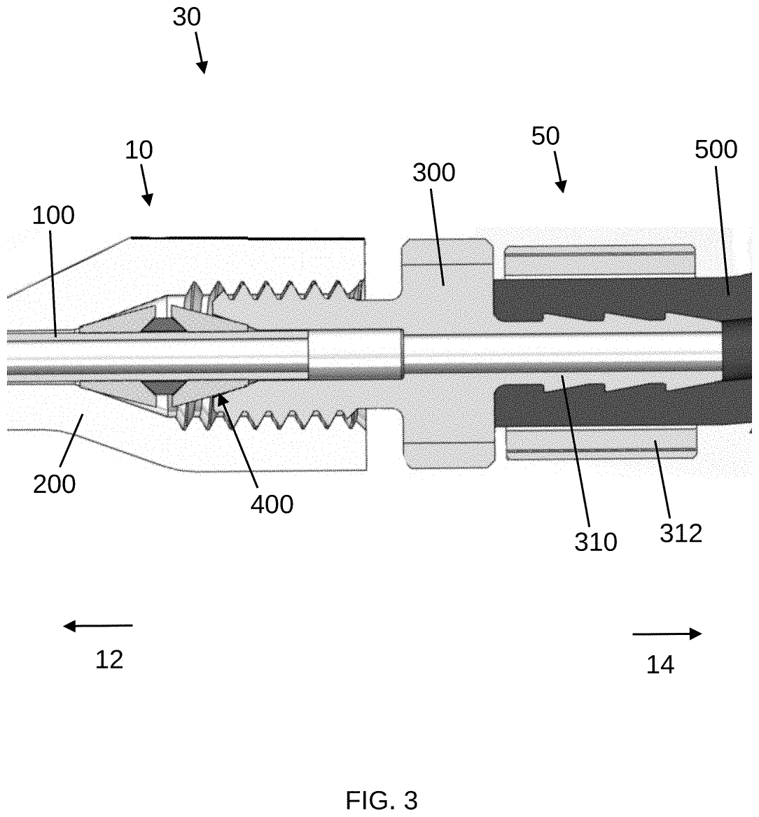

FIG. 3 is a detailed cross-sectional view of an example coupling 30, according to at least some aspects of the present disclosure. The example coupling 30 may include the example compression fitting 10 described in detailed with reference to FIG. 1. Repeated description of the various components of the example compression fitting 10 is omitted for brevity.

In some example embodiments, the coupling 30 may include a pressure-tight (e.g., sealed) connection 50 between the second threaded component 300 and a second fluid conduit 500. For example, the second threaded component 300 may include a barb fitting 310, which may be configured to engage a tube or hose comprising the second fluid conduit 500. The barb fitting 310 may include a clamp 312.

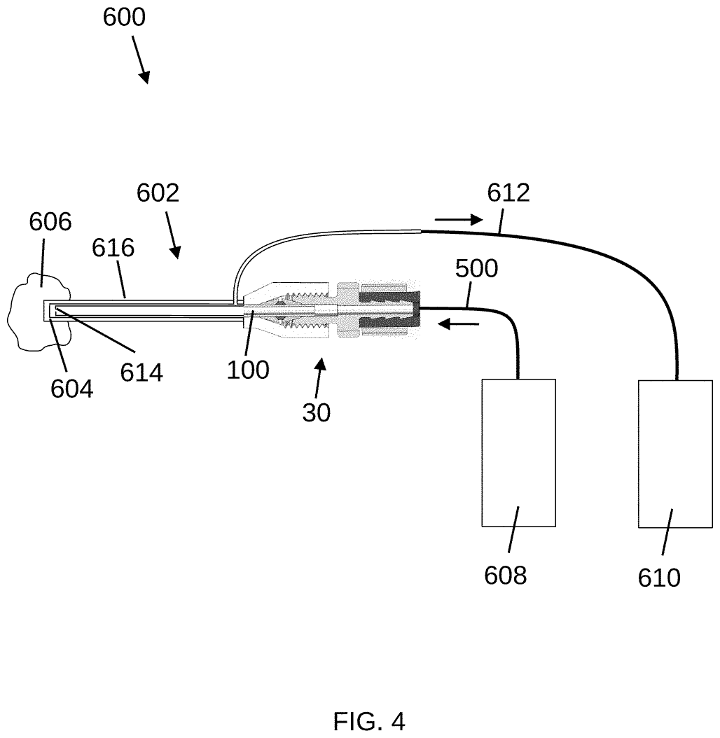

FIG. 4 is a simplified schematic view of an example cryogenic surgical system 600, according to at least some aspects of the present disclosure. The example cryogenic surgical system 600 may include a cryogenic probe 602, which may include a tissue-contacting portion 604 configured to apply extremely cold temperatures to a target tissue 606. The cryogenic probe 602 may include the example coupling 30 of FIG. 3, which may operatively connect the cryogenic probe 602 to the second fluid conduit 500. Repeated description of the various components of the example coupling 30 is omitted for brevity. The first fluid conduit 100 may act as an inlet conduit for the cryogenic probe 602, conveying the cryogenic fluid distally to the tissue-contacting portion 604. In this example embodiment, the second fluid conduit 500 may act as a cryogenic fluid supply conduit to convey cryogenic fluid between a cryogenic fluid source 608 and the cryogenic probe 602. The cryogenic probe 602 may also be connected to a cryogenic fluid receptacle 610 via a cryogenic fluid exhaust conduit 612 in fluid communication with the inner cavity 202 (see FIG. 1). In this manner, as cryogenic fluid is conveyed distally within the first fluid conduit 100, the cryogenic fluid eventually exits one or more nozzles 614 located in proximity to the tissue-contacting portion 604. By way of example, the cryogenic fluid may boil and/or decrease in pressure, resulting in a cooling (and possibly reduced temperatures) at the tissue-contacting portion. The cryogenic fluid exiting the nozzles 614 is eventually directed proximally in a counter flow over the outside of the first fluid conduit 100, which may operate to cool the cryogenic fluid traveling within the first fluid conduit 100. By way of example, the cryogenic fluid traveling proximally over the first fluid conduit 100 is contained by a shell 616 of the cryogenic probe 602, where an interior of the shell is in fluid communication with the cryogenic fluid exhaust conduit 612. In this manner, the sealed interfaces 16, 20 (see FIG. 1) may prevent leakage of cryogenic fluid proximally beyond the fitting 10 and, thus, direct all spend cryogenic fluid through the cryogenic fluid exhaust conduit 612 and into the cryogenic fluid receptacle 610.

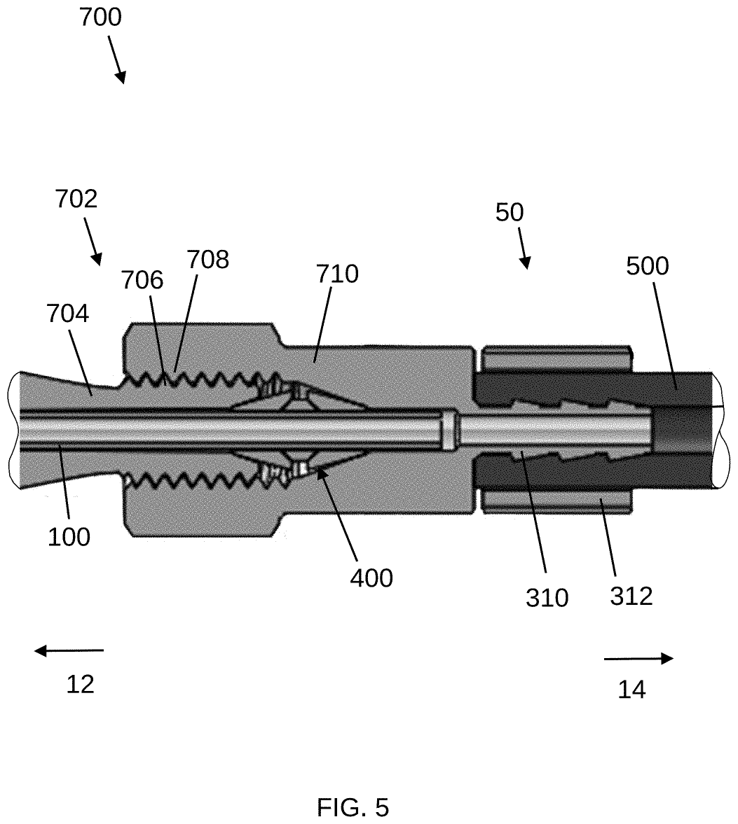

FIG. 5 is a detailed cross-sectional view of an alternative example coupling 700, according to at least some aspects of the present disclosure. The coupling 700 may include a compression fitting 702. In contrast to the coupling 30 of FIG. 3 (including the compression fitting 10 of FIG. 1), coupling 700 of FIG. 5 (including the compression fitting 702) may be configured so that a first threaded component 704 comprises an externally threaded portion 706 configured to threadedly engage an internally threaded portion 708 of a second threaded component 710. With the exception of the reversal of the externally threaded portions 304, 706 and the internally threaded portions 204, 708 on the respective first threaded components 200, 704 and second threaded components 300, 710, the description of the construction and operation of the coupling 30 and compression fitting 10 may be generally applicable to the coupling 700 and compression fitting 702 and/or the description of the construction and operation of the coupling 700 and compression fitting 702 may be generally applicable to the coupling 30 and compression fitting 10. Generally, the coupling 700 of FIG. 5 may be substituted for the coupling 30 of FIG. 3 and/or the coupling 30 of FIG. 3 may be substituted for the coupling 700 of FIG. 5, as desired. Repeated description of similar components, features, and functions is omitted for brevity.

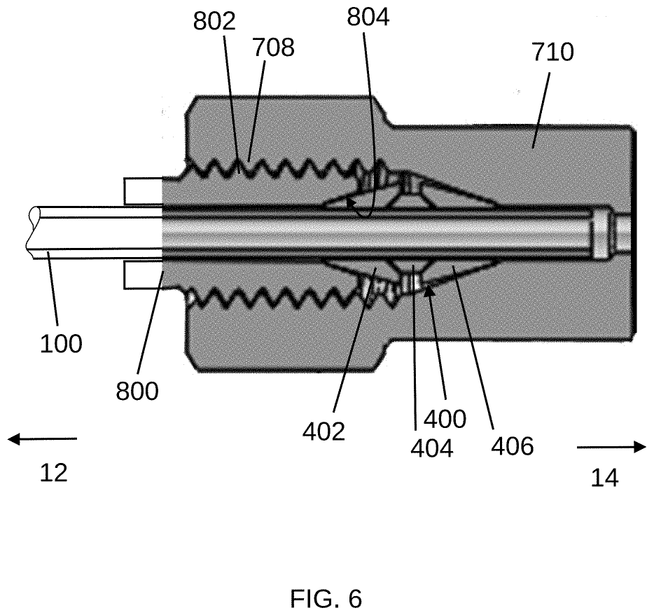

FIG. 6 is a detailed cross-sectional view of an example swaging tool 800, according to at least some aspects of the present disclosure, that may be utilized to swage one or both ferrules 402, 406 onto the first fluid conduit 100, such as prior to engagement of the first and second threaded components 704, 710. As used herein, “swaging” may refer to compressing and deforming a component to affix it to another component, creating a permanent joint. The present disclosure contemplates that swaging operations may require relatively large forces to form the desired engagement between the swaged components. In circumstances utilizing threaded components to apply the swaging forces, relatively high torques may be required to generate the desired swaging forces. Some example couplings and/or fittings according to the present disclosure may include threaded components (e.g., first and second threaded components 200, 702, 300, 710) that may not be adapted to apply torques sufficient for swaging. For example, the first threaded components 200, 702 may be constructed from plastic, such as PEEK. In some such embodiments, and in other circumstances in which it may not be desirable to utilize one or more of the threaded components 200, 702, 300 710, one or more swaging tools may be utilized to swage the ferrule set 700 onto the first fluid conduit 100. Generally, swaging tools may be configured to apply the forces necessary to conduct the swaging operation. For example, the swaging tool 800 may be constructed from stainless steel.

The example swaging tool 800 illustrated in FIG. 6 may include an externally threaded portion 802 configured to threadedly engage the internally threaded portion 708 of the second threaded component 710. The example swaging tool 800 may include an inwardly facing surface 804 adapted to engage the ferrule set 400, which may be generally similar to the corresponding feature of the first threaded component 704.

Generally, the example swaging tool 800 illustrated in FIG. 6 may be configured for use in connection with the alternative example coupling 700 of FIG. 5. Alternative example swaging tools according to at least some aspects of the present disclosure may be configured for use in connection with other couplings and compression fittings, such as coupling 30 and compression fitting 10 described above. In some such embodiments, the threaded portion of the swaging tool may include features generally similar to the corresponding features of the first threaded component 200, such as the internally threaded portion 204 and/or the inwardly facing surface 206.

Referring to FIGS. 1, 2, and 5, an example method of assembling a fitting in accordance with the instant disclosure may include one or more of the following operations. A ferrule set 400 may be positioned on a first fluid conduit 100. For example, a first ferrule 402, a tapered washer 404, and/or a second ferrule 406 may be positioned on the first fluid conduit 100, such as in a desired order. By way of example, the order may initially include sliding the first ferrule 402 over the first conduit 100, followed by sliding on the tapered washer 404 over the first fluid conduit 100, followed by sliding on the second ferrule 406 over the first conduit 100 to have an order represented as depicted in FIG. 1.

Referring to FIGS. 1, 2, 5, and 6, the ferrule set 400 may be swaged to the first fluid conduit 100 using an example method of installing a ferrule set 400 comprising one or more of the following operations. The second threaded component 300, 710 may be installed on the first fluid conduit 100, such as from the proximal end. A swaging tool 800 or a structure similar to the first threaded component 200, 704 may be installed onto the first fluid conduit 100 from the opposite end (e.g., the distal end). Thus, the ferrule set 400 may be positioned on the first fluid conduit 100 between the swaging tool 800 (or first threaded component 200, 704) and the second threaded component 300, 710. The swaging tool 800 (or the first threaded component 200, 704) may be threadedly engaged with the second threaded component 300, 710 to longitudinally and radially compress the ferrule set 400 (e.g., the first ferrule 402 and/or the second ferrule 406) into secure engagement with the first fluid conduit 100. For example, the swaging tool 800 (or the first threaded component 200, 704) and the second threaded component 300, 710 may be threaded together and torqued to a desired torque or desired number of turns.

By way of further example, where the swaging tool 800 is utilized, this swaging tool 800 may be removed from the first fluid conduit 100, followed by installation of the first threaded component 200, 704 over the first fluid conduit 100, such as from the distal end. In this manner, the first threaded component 200, 704 and the second threaded component 300, 710 may be threaded together and tightened as desired after the ferrules 402, 406 are swaged with respect to the first fluid conduit 100.

Referring back to FIGS. 3 and 5, in embodiments including a connection 50 between the second threaded component 300, 710 and the second fluid conduit 500, this connection 50 may be assembled by positioning the second fluid conduit 500, 710 over the barb fitting 310 so that one or more of the barbs engage the second fluid conduit 500. By way of further example, an interior surface of the second fluid conduit 500 is positioned against an outside surface of the barb fitting 310 so that a clamp 312 circumscribing the second fluid conduit 500 can be installed. By way of further example, the clamp 312, when installed, may be operative to compress the interior surface of the second fluid conduit 500 against the outside surface of the barb fitting 310 to form a fluid tight seal therebetween. In view of the foregoing, a closed fluid system may be created between the cryogenic fluid source 608 and the cryogenic fluid receptacle 610.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A ferrule coupling comprising:
a male fitting including a bore;
a female fitting including a bore, the female fitting sized to receive the male fitting;
a conduit sized to fit within the bores of the male fitting and the female fitting, wherein a circumferential gap exists between an exterior of the conduit and an interior surface of the female fitting demarcating the bore;
a first ferrule including dual conical surfaces and a cylindrical bore, the cylindrical bore sized to receive the conduit;
a second ferrule including dual conical surfaces and a cylindrical bore, the cylindrical bore sized to receive the conduit; and,
a washer including dual conical surfaces and a cylindrical bore, the cylindrical bore sized to receive the conduit.

2. The ferrule coupling according to claim 1, wherein:
the interior surface of the female fitting delineates a cylindrical cavity section transitioning into a conical cavity section; and,
an interior surface of the male fitting delineates a conical cavity section.

3. The ferrule coupling according to claim 2, wherein:
the conical section of the female fitting is adjacent a section having an exposed helical cavity; and,
the male fitting includes an exposed helical thread configured to be received within the exposed helical cavity and convert rotational motion between the male and female fittings into longitudinal motion between the male and female fittings.

4. The ferrule coupling according to claim 2, wherein:
the conical section of the female fitting is sized to receive the first ferrule;
a first of the dual conical surfaces of the first ferrule is configured to engage the interior surface of the conical section of the female fitting;
the conical section of the male fitting is sized to receive the second ferrule; and,
a first of the dual conical surfaces of the second ferrule is configured to engage the interior surface of the conical section of the male fitting.

5. The ferrule coupling according to claim 4, wherein:
a second of the dual conical surfaces of the first ferrule is configured to engage a first of the dual conical surfaces of the washer; and,
a second of the dual conical surfaces of the second ferrule is configured to engage a second of the dual conical surfaces of the washer.

6. The ferrule coupling according to claim 2, wherein:
the first ferrule includes a frustoconical exterior shape;
the second ferrule includes a frustoconical exterior shape;
the conical cavity section of the annual female fitting has a frustoconical shape; and,
the conical cavity section of the annual male fitting has a frustoconical shape.

7. The ferrule coupling according to claim 6, wherein:
the conical cavity section of the annual male fitting tapers in an opposite direction than does the conical cavity section of the female fitting when the male fitting is received and coupled to the female fitting; and,
the frustoconical exterior shape of the first ferrule tapers in an opposite direction than does the frustoconical exterior shape of the second ferrule when the male fitting is received and coupled to the female fitting.

8. The ferrule coupling according to claim 2, wherein:
the first ferrule includes a frustoconical interior cavity;
the second ferrule includes a frustoconical interior cavity;
the washer includes at least two exterior surfaces each having a frustoconical shape; and,
the dual conical surfaces of the washer taper in opposite directions.

9. The ferrule coupling according to claim 8, wherein:
the frustoconical interior cavity of the first ferrule tapers in the same direction as does the frustoconical shape of a first of the at least two exterior surfaces of the washer when the first ferrule and the washer are adjacent one another and both circumscribe the conduit; and,
the frustoconical interior cavity of the second ferrule tapers in the same direction than does the frustoconical shape of a second of the at least two exterior surfaces of the washer when the second ferrule and the washer are adjacent one another and both circumscribe the conduit.

10. The ferrule coupling according to claim 9, wherein the washer is wedged between the first ferrule and the second ferrule when:
the first ferrule is wedged between the conduit and the female fitting; and,
the second ferrule is wedged between the conduit and the male fitting.

11. A ferrule coupling comprising:
an annular male fitting including a bore;
an annual female fitting including a bore, the annular female fitting sized to receive the annular male fitting;
a conduit sized to fit within the bores of the annular male fitting and the annular female fitting;
a first ferrule circumscribing at least a first portion of the conduit, the first ferrule including dual conical surfaces;
a second ferrule circumscribing at least a second portion of the conduit, the second ferrule including dual conical surfaces; and,
a washer circumscribing at least a third portion of the conduit, the washer interposing the first ferrule and the second ferrule, the washer including dual conical surfaces;
wherein the first ferrule, the second ferrule, and the washer longitudinally interpose the annular male fitting and the annular female fitting;
wherein the first ferrule radially interposes the annular male fitting and the conduit;
wherein the second ferrule and the washer radially interpose the annular female fitting and the conduit.

12. A ferrule coupling comprising:
a pair of ferrules, each of the pair of ferrules including a through bore and a first external inclined surface, a first interior inclined surface, and a bore interior surface delineating the through bore, where an incline of the first external inclined surface is less than sixty degrees with respect to a longitudinal axis centered with respect to the through bore, and where an incline between of the first interior inclined surface is no greater than ninety degrees with respect to the longitudinal axis; and,
a washer configured to interpose the pair of ferrules and including a through bore, the washer including a first exterior inclined surface and a second exterior inclined surface, where the first exterior inclined surface of the washer is configured to engage the first external inclined surface of a first of the pair of ferrules, and where the second exterior inclined surface of the washer is configured to engage the first external inclined surface of a second of the pair of ferrules.

13. A method of creating a fluid tight seal across conduit sections, the method comprising:
circumscribing a first conduit with a first ferrule, a first washer, and a second ferrule, where the first ferrule comprises dual conical surfaces tapering in the same direction, and where the second ferrule comprises dual conical surfaces tapering in the same direction;
compressing the first ferrule between the first conduit and a first fitting to form first seals, where a first of the first seals is between the first ferrule and the conduit, and where a second of the first seals is between the first ferrule and the first fitting;
compressing the second ferrule between the first conduit and a second fitting to form second seals, the second fitting including at least a portion of a second conduit, where a first of the second seals is between the second ferrule and the conduit, and where a second of the second seals is between the second ferrule and the second fitting; and,
coupling the first fitting to the second fitting to maintain the first seals and the second seals.

14. The method of claim 13, wherein:
the act of compressing the first ferrule between the first conduit and the first fitting to form first seals includes swaging the conduit at a first circumferential location;
the act of compressing the second ferrule between the first conduit and the second fitting to form second seals includes swaging the conduit at a second circumferential location; and,
the first circumferential location is longitudinally spaced along a length of the conduit from the second circumferential location.

15. The method of claim 14, wherein:
compressing the first ferrule between the first conduit and the first fitting includes longitudinally repositioning a washer, including dual frustoconical surfaces, along an exterior of the first conduit; and
longitudinally repositioning the washer includes longitudinally repositioning the second ferrule along the exterior of the first conduit.

16. The method of claim 15, wherein longitudinal repositioning of the washer and the second ferrule is the result of longitudinal repositioning of the first fitting with respect to the second fitting.

17. The method of claim 16, wherein:
the first fitting and the second fitting are configured to threadably engage one another; and,
longitudinal repositioning of the first fitting with respect to the second fitting is the result of rotation of the first fitting with respect to the second fitting when the first and second fittings are threadably engaged.

18. A cryogenic surgical system, comprising:
the coupling of claim 1;
a cryogenic probe, the cryogenic probe comprising the conduit;
a cryogenic fluid source; and
a second conduit extending between the cryogenic fluid source and the coupling.

19. A cryogenic surgical system, comprising:
the coupling of claim 11;
a cryogenic probe, the cryogenic probe comprising the conduit;
a cryogenic fluid source; and
a second conduit extending between the cryogenic fluid source and the coupling.

20. A cryogenic surgical system, comprising:
the coupling of claim 12;
a pair of fittings housing the couplings;
a cryogenic probe in sealed fluid communication with a conduit extending through the coupling;
a cryogenic fluid source; and
a second conduit extending between the cryogenic fluid source and the coupling.
